(19) United States
(12) Patent Application Publication
(10) Pub. No.: US 2010/0189752 A1
(43) Pub. Date: Jul. 29, 2010

(54) PELLETED MINERAL SUPPLEMENT FOR LAND ANIMALS

(76) Inventors: Haig H. Kassouni, Grand Rapids, MI (US); Armen S. Kassouni, Grand Rapids, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 12/754,184

(22) Filed: Apr. 5, 2010

Related U.S. Application Data
(63) Continuation-in-part of application No. 12/021,618, filed on Jan. 29, 2008.

(51) Int. Cl. A61K 9/00; A61K 33/14; A61K 33/42; A23K 1/16; A23K 1/18

(52) U.S. Cl. 424/400; 424/680; 424/678; 424/602

(57) ABSTRACT

A pelleted mineral supplement for land animals is provided. Each of the pellets is a cohesive mass of mineral components that when consumed by a land animal promotes the health and/or growth of the animal, and each of the cohesive masses has a largest dimension less than 0.5 inches. In certain aspects of the invention, the pelleted mineral supplement can be combined with a conventional animal feed to provide a mineral-supplemented animal feed.

### CROSS-REFERENCE TO RELATED APPLICATION

This application is a continuation-in-part of U.S. patent application Ser. No. 12/021,618 filed Jan. 29, 2008, entitled MINERAL LICK, the entirety of which is incorporated herein by reference.

### FIELD OF THE INVENTION

This invention relates to mineral supplements for land animals, especially land based wildlife and game. The term “land animals” is intended to include birds and fowl. The term “wildlife” as used herein refers to wild or game land animals.

### BACKGROUND OF THE INVENTION

It is generally believed that land animals, especially deer and other members of the family Cervidae, are attracted to salts and other mineral nutrients, and that mineral supplementation, especially of biovailable phosphorous and calcium, promotes or at least facilitates antler development in males and may be beneficial for lactating females, generally improving the health and strength of a herd. Hunters, wildlife photographers and other nature enthusiasts have used artificial salt or mineral licks to attract deer and other mammals belonging to the family Cervidae, and to promote the health of these animals.

Commercially available products for preparing an artificial salt or mineral lick for members of the family Cervidae have been limited to large bricks (e.g., 4 to 50 pound bricks having dimensions up to about 8 inches by 8 inches by 11 inches), and bagged powders. Typically, the bricks are simply placed on the ground and allowed to slowly dissolve by exposure to humidity and precipitation. Bagged powders may be poured directly onto the ground, and optionally mixed into the soil, or dissolved and/or suspended in water that is subsequently poured onto the ground.

### SUMMARY OF THE INVENTION

This invention provides a pelleted mineral supplement for land animals that may be easily dispensed and metered at a selected site of application, or which may be blended or otherwise combined with an animal feed to provide a mineral-supplemented animal feed. The pelleted mineral supplements of this invention include a cohesive mass of mineral components that when consumed by a land animal promote the health and/or growth of the animal, and wherein the cohesive mass has a largest dimension less than 0.5 inches.

The invention also provides a mineral-supplemented animal feed which includes a combination of an animal feed containing a source of proteins, fats, and carbohydrates, and a pelleted mineral supplement in the form of a cohesive mass of mineral components that when consumed by a land animal promote the health and/or growth of the animal, and wherein the compressed cohesive mass has a largest dimension less than 0.5 inches.

These and other features, advantages and objects of the present invention will be further understood and appreciated by those skilled in the art by reference to the following specification and claims.

### DETAILED DESCRIPTION OF PREFERRED EMBODIMENTS

The expression “cohesive mass” as used herein refers to a mass of solid material having a suitable hardness and a relatively low friability such that it will not easily break, disintegrate or otherwise breakdown during normal handling, so that it can be carried, such as in a person's pocket, to a site of application or easily disbursed (e.g., blended or mixed) into animal feedstuff The cohesive mass of the invention is similar to a compressed pharmaceutical or nutritional tablet, except for its size, and may be prepared in a manner similar to the manner in which pharmaceutical and nutritional compressed tablet dosage forms are prepared. Alternatively, conventional pelleting equipment may be employed to form suitable pelleted material.

The term “pelleted” refers to any process that can be employed to make a cohesive mass in accordance with the invention, and the pelleted material may have any suitable shape, such as spherical, cylindrical, flakes, etc.

The cohesive mass of the invention may comprise or consist essentially of mineral components and optionally small amounts of other components. A cohesive mass consisting essentially of mineral components refers to a cohesive mass that contains either none or unsubstantial amounts of other nutritional materials (e.g., fats, proteins, and carbohydrates), but which may contain small amounts of additives such as colorants (e.g., food coloring), flavorants and/or scents (e.g., apple, corn or molasses), attractants (e.g., estrous/urine scent), and excipients, such as binders (e.g., starch or starch derivatives), disintegrants (e.g., sodium starch glycolate), and lubricants (e.g., stearic acid). Unsubstantial amounts of other nutritional materials are amounts that constitute less than 10% of the mass of the cohesive mass in the case of starches and starch derivatives or other materials used as binders and/or disintegrants, with only trace impurities of other nutritive ingredients, and about 2% or less of a lubricant based on the mass of the cohesive mass. Desirably, the components are selected to provide an extremely long or indefinitely long shelf life. This is achieved by excluding or minimizing organic nutritional components.

The expression “mineral nutrients” and the expression “mineral components” refer to the source materials that provide biovailable mineral nutrients.

The antlers of members of the family Cervidae are typically composed of about 20% calcium and 10% phosphorous by mass. Accordingly, it is believed that the dietary needs of Cervidae species are best satisfied with a diet containing calcium and phosphorous in a mass ratio of about 2:1. Cervidae species also require sodium in their diet. A suitable cohesive mass in accordance with the invention consists of mineral components that may contain from about 30% to about 50% sodium chloride, about 10% to about 20% calcium chloride, about 40% to about 50% dicalcium phosphate, and up to about about 2% of a tablet lubricant, and optional trace amounts of other mineral nutrients. A composition comprising or consisting essentially of mineral components and tabletting aids may contain sodium chloride, calcium chloride and dicalcium phosphate in the above proportions, with the percentages adjusted downwardly based on the addition of optional tabletting aids, and/or trace amounts of additives (e.g., colorants, flavorants, scents, attractants, etc.). Other mineral sources may be used rather than calcium chloride, dicalcium phosphate, and sodium chloride, if desired. For example, other sources of calcium include calcium sulfate and calcium citrate. Other sources of phosphate include dibasic potassium phosphate and dibasic sodium phosphate (which is also an alternative source for sodium). Preferably, when such alternative mineral sources are used, they are employed in relative amounts to achieve an elemental calcium to phosphorous mass ratio of about 2:1, and preferably an elemental sodium to calcium mass ratio of from about 1:1 to about 1.5:1.

The compositions used to prepare the cohesive mass of the invention may optionally contain trace amounts of other dietary mineral supplements to provide nutritive amounts of zinc, magnesium, sulfur, copper, iodine, cobalt, and/or selenium. Suitable sources of these dietary supplements are well-known and include, for example, zinc ascorbate, zinc citrate, zinc malate, zinc succinate, magnesium ascorbate, magnesium aspartate, magnesium citrate, sodium sulfate, cupric oxide, copper gluconate, copper sulfate, potassium iodide, sodium iodide, cyanocobalamin, sodium selenate, and sodium selenite. Suitable trace amounts of these dietary mineral supplements are known and can be selected, if desired, by reference to the literature. However, it is expected that the total amount of these trace mineral supplements will not exceed 1 or 2% by mass of the cohesive mass.

In addition to the mineral components, the cohesive masses of this invention may contain conventional processing aids such as binders, disintegrants, and lubricants. Examples of binders that may be employed include microcrystalline cellulose, starch, and starch derivatives. Such binders may be employed in generally any suitable amount, typically about 10% or less by mass to minimize the cost of the cohesive mass and to minimize the amount of non-nutritive ingredients in the cohesive mass. Examples of disintegrants that may optionally be employed, typically in relatively small amounts (such as about 5% or less by mass), include starch and sodium starch glycolate. Suitable lubricants used to prevent material from sticking to dies and punches or other equipment employed to prepare cohesive masses from powders or granules include fatty acids, such as stearic acid, and metal salts of fatty acids, such as magnesium stearate, which may be used in an amount of, for example, from about 0.5% to about 2.5% by mass.

Colorants (e.g., food coloring), scents, flavorings and attractants (e.g., estrous/urine scent) may be added to the mineral components in relatively trace amounts (e.g., typically less than 1% by mass of each) prior to pelleting the mineral components and optional tabletting aids into a cohesive mass.

The cohesive mass of the invention can be prepared using a suitably sized and shaped die and punch with a hydraulic press capable of applying sufficient pressure to a powdered mass deposited between the die and punch to convert the powdered mass into a cohesive mass having suitable hardness and a suitably low friability to facilitate handling without breakage or loss of material. Suitable compression forces range from about 5,000 to about 10,000 psi. Alternatively, various other processing techniques may be employed to form beads, spheres, cylinders, flakes, or other pelleted cohesive masses.

An appropriate size for the maximum dimension of the pelleted mineral supplements or cohesive masses of this invention is less than 0.5 inches, and more preferably less than 0.3 inches.

The cohesive mass of mineral components in accordance with the invention can be formed into generally any shape. For example, briquette, cubic or rectangular shapes can be made. However, a preferred shape is a cylinder, disk or puck shape having a diameter less than 0.5 inches, with a typical cohesive mass of mineral components in accordance with the invention having a largest dimension of about 0.1 to about 0.3 inches.

In accordance with certain other aspects of the invention, the cohesive mass of mineral components may be color-coded based on recipe. The cohesive mass of mineral components in accordance with the invention may also be used as a carrier for other types of animal products, such as contraceptives or specialized vitamins.

Because certain mineral components may be hygroscopic, it may be desirable to pack the compressed cohesive mass or masses in accordance with the invention in a moisture resistant package (e.g., plastic container or wrap having a moisture barrier property)

The pelleted mineral supplements of this invention may be combined with an animal feed containing a source of proteins, fats, and carbohydrates to provide a mineral supplemented animal feed. The proportion of animal feed to pelleted mineral supplement may be adjusted as desired based on the desired level of supplementation and the amount, if any, of mineral nutrients in the animal feed. However, typical proportions of animal feed to pelleted mineral supplemental may range from about 50:1 to 2000:1 on a weight basis. Suitable animal feed having an appropriate protein, fat, and carbohydrate content for promoting the health and growth of a selected land animal are generally well-known and commercially available. The present invention is not directed to improvements of animal feed, except in combination with the pelleted mineral supplements.

The above description is considered that of the preferred embodiments only. Modifications of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the doctrine of equivalents.

The invention claimed is:

1. A pelleted mineral supplemental for land animals, comprising:
a cohesive mass of mineral components that when consumed by a land animal promotes the health and/or growth of the animal, the cohesive mass having a largest dimension less than 0.5 inches.

2. The mineral supplement of claim 1, wherein the cohesive mass has a largest dimension less than 0.3 inches.

3. The mineral supplement of claim 1, wherein the cohesive mass has a cylinder shape.

4. The mineral supplement of claim 1, wherein the cohesive mass has a briquette shape.

5. The mineral supplement of claim 1, wherein the mineral components include a source of sodium, calcium, and phosphorous.

6. The mineral supplement of claim 1, wherein the mineral components include sodium chloride, calcium chloride, and bicalcium phosphate.

7. The mineral supplement of claim 1, packed in a moisture-resistant package.

8. The mineral supplement of claim 1 which includes a scent, attractant, flavorant, or a mixture of two or more thereof.

9. The mineral supplement of claim 1, wherein the cohesive mass has a largest dimension of from 0.1 inches to 0.3 inches.

10. A mineral-supplemented animal feed, comprising:
a combination of an animal feed containing a source of proteins, fats, and carbohydrates; and
a pelleted mineral supplement in the form of a cohesive mass of mineral components that when consumed by a land animal promotes the health and/or growth of the animal, the compressed cohesive mass having a largest dimension less than 0.5 inches.

11. The mineral-supplemented animal feed of claim 10, wherein the cohesive mass has a largest dimension less than 0.3 inches.

12. The mineral-supplemented animal feed of claim 10, wherein the cohesive mass has a cylinder shape.

13. The mineral-supplemented animal feed of claim 10, wherein the cohesive mass has a briquette shape.

14. The mineral-supplemented animal feed of claim 10, wherein the mineral components include a source of sodium, calcium, and phosphorous.

15. The mineral-supplemented animal feed of claim 10, wherein the mineral components include sodium chloride, calcium chloride, and bicalcium phosphate.

16. The mineral-supplemented animal feed of claim 10, packed in a moisture-resistant package.

17. The mineral-supplemented animal feed of claim 10 which includes a scent, attractant, flavorant, or a mixture of two or more thereof.

18. The mineral-supplemented animal feed of claim 10, wherein the cohesive mass has a largest dimension of from 0.1 inches to 0.3 inches.
